(19) United States
(12) Patent Application Publication
(10) Pub. No.: US 2020/0388387 A1
(43) Pub. Date: Dec. 10, 2020

(54) SYSTEM OF MONITORING, PROCESSING, ANALYSIS AND STORAGE OF THE PHYSIOLOGICAL SIGNS FOR THE RECOGNITION OF EMOTIONS

(71) Applicant: AFFECT SENSE, S.C., VERACRUZ (MX)

(72) Inventors: GENARO REBOLLEDO MENDEZ, VERACRUZ (MX); YECELY ARIDAI DIAZ BERISTAIN, VERACRUZ (MX); NORBERTO REBOLLEDO MENDEZ, VERACRUZ (MX); JOVAN DAVID REBOLLEDO MENDEZ, VERACRUZ (MX)

(73) Assignee: AFFECT SENSE, S.C., VERACRUZ (MX)

(21) Appl. No.: 16/545,016

(22) Filed: Aug. 20, 2019

(30) Foreign Application Priority Data
Jun. 6, 2019 (MX) MX/A/2019/006648

(51) Int. Cl. G16H 50/20; G06N 5/04; G16H 10/65; G16H 15/00; G16H 50/30; G09B 19/00; A63F 13/212; A63H 3/00

(52) U.S. Cl. CPC G16H 50/20; G06N 5/04; G16H 10/65; G16H 15/00; A63H 3/02; G09B 19/00; A63F 13/212; A63H 3/003; G16H 50/30

(57) ABSTRACT

The invention relates to a system, a method and a computer readable medium for monitoring, processing, analyzing and storing physiological signals for the recognition of emotions of at least one user, the monitoring is performed by at least one sensor of a plurality of sensors, monitoring signals corresponding but not limited to the heart rate, temperature, neuronal activity, electro-dermal activity, among others, so that after performing the processing of the signals, the emotional state of the user can be determined and with this information can modify the operation of different devices, which can be a car of different type, for when the user is a driver, a video game, for when the user is playing, an ATM, for when the user is going to make use of it, a toy, an appliance, home automation device, or any device or object that can be connected to the Internet.

### TECHNICAL FIELD

The present invention belongs to the technical field of computer systems. Particularly in the technical field of data processing and even more in particular in the area of emotional recognition systems.

### BACKGROUND

Because of the increase in the use of technology in our society and the lack of mechanisms to monitor the emotions of users, an invention with a title system of monitoring, processing, analysis and storage of physiological signals for the recognition of emotions is proposed.

It is intended to protect said invention through the present application because it is a novel proposal that allows processes, products and/or services of third parties to have emotional information of the person who is interacting with these processes, products and/or services. The proposed system has the novelty of not only detecting emotional states but also has the facility to store such information to have a historical record of the user's emotional behavior in front of a process, product and/or service.

In the literature there are some proposals for the recognition of emotions that have to do with the present application but are more specific or used means that differ from those in the present application. For example, U.S. Pat. No. 8,180,638 B2 describes a methodology for classifying voice-based emotions. Similarly, U.S. Pat. No. 8,538,755 B2 and US 20080052080 A1 also use the voice to recognize emotions. U.S. Pat. 20120143022 A1, like the present application, proposes a system for the recognition of emotions using physiological signals different from those set out in the present application.

Although there are other patents dedicated to the recognition of emotions, the invention presented in this proposal has the particularity of not only describing mechanisms for monitoring, processing, analysis and storage of physiological signals to detect emotions, but it also offers a system to provide the detected emotions to processes, products and/or services of third parties that benefit from the emotional information of the users. The pieces, details and characteristics of this methodology for the recognition of emotions are clearly shown in the following description and in the accompanying drawings.

### OBJECT OF THE INVENTION

The system of monitoring, processing, analysis and storage of physiological signals for the recognition of emotions of users, consists in allowing other inventions, applications or devices including those based on the web and users, store and track their emotions. In a modality it implies that the applications, objects, devices or services, as well as the user himself, always access the emotional states generated by the users. In an operation mode, a bio-transmitter (10) reads information, through a user interface (11) that is connected to a group of sensors in at least one user of a plurality of users that is monitored, while the information that is collected is sent to a portable electronic telecommunications device or mobile device which collects information related to the bio-signals of a user, while the user performs a particular situation, for example driving a vehicle.

In a further embodiment, readings of a Neuronal Activity (NA) of a user are read, read through a neuronal activity reading device such as but not limited to a sensor (04), Electro Dermal Activity (EDA) is read using, for example, but not limited to a device reading electrothermal activity, sensor (02) and heart rate (HR) read by a heart rate reader device (03), are collected by a user interface (11) of a portable or mobile electronic telecommunications device, wherein the bio-transmitter encodes said signals.

The coding of the bio-signals made in the bio-transmitter (30) is encrypted and sent via internet to an online processing system (20) located in a centralized server and permanently connected to the Internet. A receiver (41) is a module designed to perform the step of receiving the encrypted information, so that the receiver (41) contains logic circuits and also contains the logic necessary to decrypt the information sent by the bio-transmitter (10) and to separate the information into components, which contains the data of EDA, HR and NA.

Said online processing system (20) which is located on at least one server, is responsible for carrying out all signal processing in order to determine the emotional state of the monitored user, which may be a plurality of users monitored at the same time. time, so that finally by means of a mobile device and in which there is a transmitter of emotions (30) is responsible for sending the data by means of a user interface (31) to the necessary elements that make use of the information that It contains the emotional state of the user, so that at least one device, object application or service performs the process assigned for that condition.

### BRIEF DESCRIPTION OF THE FIGURES

The objects, features and advantages of the present invention are best appreciated when considered in conjunction with the accompanying drawings in which:

FIG. 1: Illustrates the flow chart showing the general operation of the invention.

FIG. 2: Illustrates the minimum block diagram of the invention.

FIG. 3: Illustrates a general block diagram of the invention.

FIG. 4: Illustrates an embodiment of the invention implemented in a vehicle.

FIG. 5: Illustrates a further embodiment of the invention implemented in video games.

FIG. 6: Illustrates an additional modality implemented in toys.

FIG. 7: Illustrates a modality implemented in ATMs.

### DETAILED DESCRIPTION OF THE INVENTION

The present technical description is developed based on details of the exemplary embodiments, as illustrated in the appended figures, specifying that the modalities described below are presented as examples and do not represent all the implementations contained in the present description, so the terminologies used in this specification are only to describe particular modalities without limiting the present description.

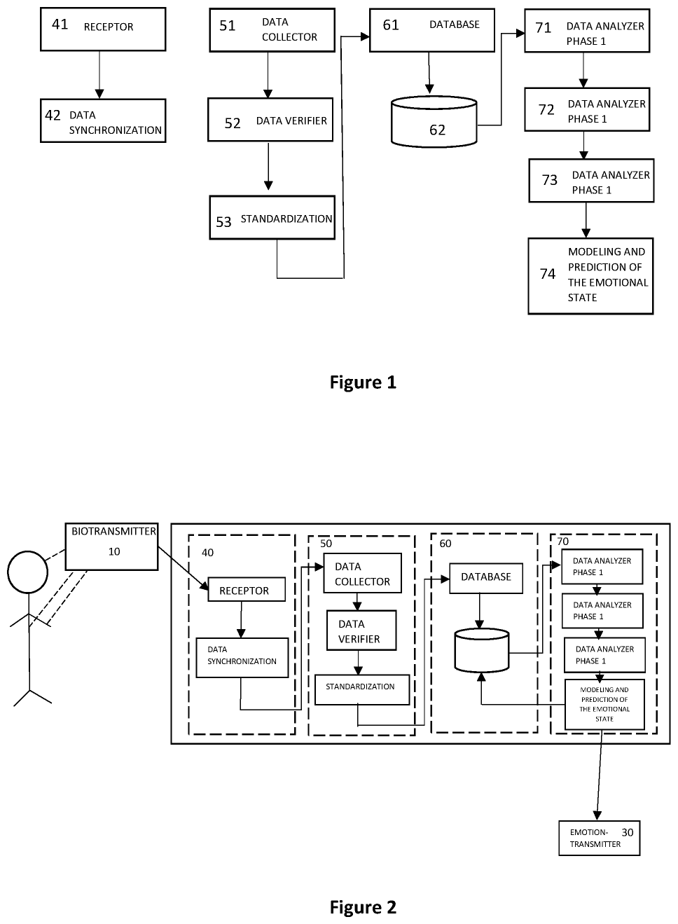

FIG. 1 shows the diagram of the process, which is developed in the present invention to carry out the different stages involved in carrying out the monitoring, as well as the processing of physiological signals, for the automatic recognition of said signals, so that in said process, in the step (41) the data reception process is performed, so that later in the step (42) a synchronization of the obtained data is carried out, so that after synchronizing the data, it is performs the step (51) in which the data is collected to perform a verification thereof, this verification being carried out in step (52), and then the data is normalized in step (53), to subsequently send the data to a database in steps (61) and (62), and subsequently the data analysis stage is carried out, comprising of three stages (71) for phase 1, (72) for phase 2, (73) for phase 3 and in stage (74) is performed a modulation and prediction of the emotional state of the person to whom the data of the bio-signals is being processed.

The description that follows is made considering both FIG. 1 and FIG. 2, since the diagram of FIG. 1 is shown considering the blocks shown in FIG. 2, to show the stages that make up each block of the invention.

As seen in FIG. 2, the bio-transmitter (10) is responsible for sending the signals of the readings taken to at least one user, but not limited to a user, since the reading of data can be performed to multiple users , these data are sent through the Internet to a server or main computer, where the module (20) is located which is an online processing system, so the online processing system when carrying out the processing of the data of the bio-signals, sends said information by means of an emotion transmitter module (30) for its later use. Next, additional details that identify FIGS. 1 and 2 are described.

### Bio-Transmitter

The bio-transmitter (10) comprises a user interface in the form of applications for mobile telecommunications electronic devices that constantly monitor the user and collect the bio-signals by means of one or multiple sensors (01, 02, 03, 04), which are in a user or multiple users in a specific situation and which, in addition to collecting the bio-signals, compresses and transmits said bio-signals to the online processing system (20) through the Internet.

The monitored bio-signals that comprise, but are not limited to, electro-dermal activity (02), heart rate (03), blood pressure (03), temperature (03), neuronal activity (04) and other physiological data. The connections between the bio-transmitter (10) and the biosensor are established wirelessly, and the user can increase or reduce the number of bio-signals used (by increasing or reducing the number of devices capable of reading said information) depending in their preferences and their availability.

To allow these connections, and as can be seen in FIG. 2, the preferred embodiment of the bio-transmitter (10) is by means of a user interface (11) in mobile devices, preferably, but not limited to, intelligent third-party cell phones companies By doing this, the bio-transmitter is close enough to the bio-sensors of third-party companies that it has constant monitoring of the physiological changes that the user has in real time. At the same time, the preferred implementation of the bio-transmitter (10) of the user interface found in smartphones, which use Internet connections to communicate said bio information via the Internet to the Online Processing System (20).

### Online Processing System

The steps that are developed in the online processing system (20) are performed by means of a processor, these stages comprise subprocesses for a) receiving the bio-signals by means of the bio-signal receiver (40); b) clean bio-signals to produce information (50); c) saving the information in a data store (60); d) analyze the information to give an estimate of the current emotions of the users and a prediction of the emotions (70) of the users in the near future. These emotions and the prediction of emotion are communicated to the transmitter of emotions (30) and to the system of consultation of emotions, which includes the necessary logic to consult, obtain and visualize emotional states and their predictions.

The online processing system (20) comprises the use of user interface components (which may be, but not limited to a front-end), an interface for communication and data management (which may be, but not limited to). a middle-ware) and an interface for the processing and storage of data (which may be, but not limited to a back-end) and other modules and services, in addition to the system that manages the database to receive and clean the bio-signals turning them into information about emotions and emotional predictions for that user in some specific activity.

In all preferred embodiments, the online processing system (20) constantly gathers new information from one or more users, establishes the emotional state (71) for a particular situation or circumstance and produces a prediction (72) for a user and for a particular situation or circumstance.

### Bio-Signal Receiver

The block (40) comprises the step to receive bio-signals from the bio-transmitter (10) by means of a signal receiver module (41) and in the data synchronization module (42) said signals are synchronized in preparation for phases Subsequent to the online processing system (20), the processing performed on the bio-signals until before this stage is only with respect to the adaptation of the bio-signals to be able to be transmitted from the bio-transmitter (10) to the online processing (20), therefore, the receiver (41) receives the raw signals, that is, without being processed.

The way of carrying out the communication of the bio-transmitter (10) with the bio-receiver of signals (40) wirelessly through the Internet. The wireless element of these connections allows the automatic recognition, storage and transmission of the user's emotions in such a way that the user performs the routine activities of his normal life. This communication is made by means of a user interface that is in the wireless element and where the wireless element is, but is not limited to a smartphone, being able to be any mobile device.

The mobile device is connected to the receiver (41) through communication and encryption protocols that allow the secure reception of the bio-signals transmitted by the bio-transmitter (10) and originated in the body of the user. The bio-signal receiver is located on a server and is not part of the user interface, but its connection to the bio-transmitter (10) and the user interface represents the input to the Online Processing System (20).

The receiver (41) comprises the logic necessary to read the bio-signals received remotely from the bio-transmitter (10) to classify them according to their origin. The origin of the bio-signals includes but is not limited to heart rate, neuronal activity, electro-dermal activity, blood pressure and other physiological signals relevant to the recognition of emotions, said logic basically responds to two functionalities consisting of 1) reception constant of bio-signals originated in the bio-transmitter (10) and transmitted through the Internet; and 2) analysis of the bio-signals so that similar bio-signal packets are passed to the information synchronization thread (42).).

After the receiver stage (41) has classified the received bio-signals, the data is sent to the data synchronization module (42), which comprises the logic necessary to organize the bio-signals received by the receiver module (41) to capture a current state of the user based on his physiological reactions, in such a way that, the organized data of the bio-signals originally in different time scales, are expressed in a homogeneous time scale that reflects a unique and constant change through time for each user.

### Bio-Signal Cleaner and Information Producer

The bio-signal cleaner and information producer (50) comprises of the modules for collecting, by means of the bio-signal collector module (51), verifying, by means of the bio-signal verifier module (52) and normalizing, by means of the bio-signaling normalization module (53) the synchronized bio-signals in the data synchronization module (42), this is done for each user to which the respective signals are being taken, in this way information valid for that user, that at the end of this stage said data are ready to be stored in the module (60), processed in the module (70) and transmitted in the module (30) which are the subsequent processes of the processing system online (20).

The bio-signal collector (51) contains the necessary logic to organize again the bio-signals that have already passed the stage of the data synchronization module (42), receiving the bio-signals in real time, in this module (51) , the stage of organization of the data, unlike that carried out in step (42), is performed in the same time line to be processed by the bio-signal verification module (52).

Once the data of the bio-signals are sent to the bio-signal verification module (52) and which comprises the necessary logic that verifies the bio-signals received from the bio-signal collector (51), so that the module (52) comprises the step of verifying that the received information is significant and sufficient to be normalized.

When the module (52) finishes its verification, the data is sent to the module (53), where the normalization stage is carried out, so it contains the logic required for said function, organizing the data of the bio-signals in information packages and verify that the information contained in the data is meaningful information, that is to say that it consists of a complete series of information associated with a Unique User Identifier (UUI) that is associated with bio-signals that are being processed, and ensure that these bio-signals are packaged with such meaningful information.

### Storage of Data in a Data Warehouse

The data storage (60), comprises of the logic necessary to store valid information in a database using standardized database protocols, the storage of data in a database (60) is a module of the processing system in line (20) which is made up of two blocks, which corresponds to the data storage system (61) and the storage of the data in the database (62).

The data storage system (61) is composed of the necessary logic that allows to classify and organize the information of the processed data from the standard bio-signals and their unique identifiers of users (UUI) originated in the module (50) for said data to be stored in the information repository (62). Likewise, they also receive data that come from the module (70), also producing in the module (60) the queries required by the system of consultation of emotions (80). This module is also responsible for communicating information organized to the subprocess of information analysis to estimate emotions and predictions (70).

It is also mentioned that, since the module (60) is part of the module (20) and the bio-transmitter (10) is permanently connected to the server where the module (20) is located, it constantly receives information comprising the data of the bio-signals of the monitored users, these data, as already mentioned, contain their unique identifiers of UUI users associated and produced by the signaling standardization module (53), and with said identifiers, the administrator of the database can classify said bio-signals to find a place in the existing structure in the database, therefore the unique identifiers of UUI users are a unique identification key of the information associated to different users.

The information repository (62) comprises logic necessary for writing and obtaining database information capable of receiving information packets comprising the data of the standard bio-signals and associated information originating in the information storage system (61) and in phase 2 of data analyzer (72). The information packages include the data of the bio-signals and the unique identifiers of users (UUI) originating in the storage system (61) and an emotional state resulting from phase 1 of the data analyzer (71) and prediction result of phase 2 of the data analyzer (72), the latter belonging to the block of information analysis to estimate emotions and predictions (70). The information packets are organized by the information repository (62) in an appropriate structure within the database. The deposit of the information (62) also comprises the logic for the recovery of data managing the queries from the phase 1 of analysis of the information (71) and the system of consultation of emotions.

In one embodiment, the data store (62) comprises the logic necessary to store the information in a tangible physical medium readable by computer, as, for example, but not limited to a magnetic medium, USB memory, hard disk, CD and others, as well as in a cloud service (Amazon WebServices, for example). Said information comprises the data of the bio-signals originated in the stage of normalization of bio-signals (53) and phase 2 of the data analyzer (72) both organized considering the unique user identifier (UUI). In a preferred embodiment, the bio-signals transmitted by the signaling normalization stage (53) are associated with an emotional projection emitted in phase 2 of the data analyzer (72). The emotional projection is also associated with the unique user identifier (UUI). In a further embodiment, the data store (62) also has the necessary logic to process queries originating in the emotion consultant system to provide a mechanism to visualize the information, which may be, but not limited to, an LCD screen, monitor , television, mobile device, among others, said emotional consultant system visualizes the data of bio-signals, past, current and forecasted emotions.

### Information Analysis to Estimate Emotions and Predictions

This block basically comprises two phases of modeling the information to estimate the current emotions of the users, which is done in the stage of the phase 1 of the data analyzer (71), and a prediction based on these emotions and on the history of the user, which is done in stage 2 of the data analyzer (72). This block includes the necessary logic based on artificial intelligence to provide a user (identified with its corresponding unique identifier of UUI users) with an emotional state based on the bio-signals as they were received from the data warehouse (62) and to provide a prediction given said emotional state and past emotional states.

The analysis of the information to estimate emotions and predictions (70) includes the logic necessary to associate the data of the bio-signals as they were received from the data warehouse (62) and a unique user identifier (UUI) to a) estimate the current emotional state performed in phase 1 of the data analyzer (71) and to b) produce a forecast based on that emotional state that is performed in phase 2 of the data analyzer (72).

About the phase 1 information analyzer (71), it has the necessary logic to perform artificial intelligence algorithms by means of at least one processor to retrieve the data from the bio-signals and unique identifiers of users (UUI) from the warehouse. of data (62) to associate this information with an emotional state. In the step of the phase 1 of the data analyzer (71) additionally comprises the logic necessary to communicate the data of said emotions to the stage of phase 2 of the data analyzer (72).

In the same way, the stage of phase 2 of the data analyzer (72) comprises the logic necessary to perform artificial intelligence algorithms in at least one processor to provide a prediction of an emotion given two sets of information, which correspond a: a) the emotion provided in the stage of phase 1 of the data analyzer (71); and b) past emotions, as they are received from the data store (62) and associated to a particular user by means of the unique user identifier (UUI). Likewise, in the stage of phase 2 of the data analyzer (72) comprises the logic necessary to communicate the emotional prediction to the transmitter of emotions (30) for the processing of emotional information, and to the data warehouse (62) for storage.

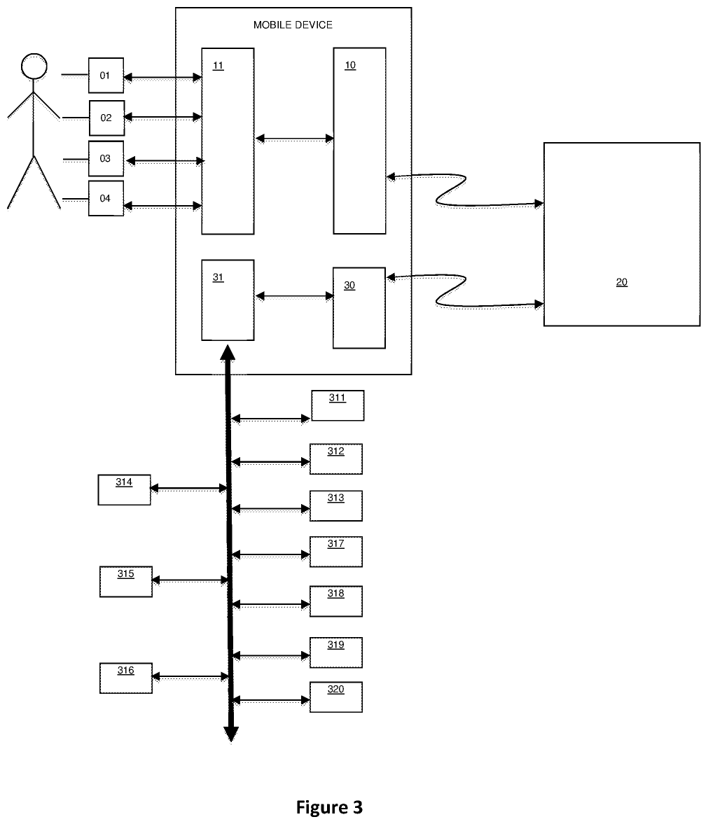

The information transmitted from the information analysis system for estimating emotions and predictions (70) to the emotion transmitter (30) represents the output of the online processing system (20). This output is transmitted via the Internet to a preferred mode of the emotion transmitter (30) comprising a user interface (31) in a portable electronic telecommunication device. The output provided by the online processing system (20) comprises past, present and future emotional states allowing other applications (314, 315, 316 of FIG. 3) or objects (311, 312, 313, 317, 318, 319, 320 of FIG. 3) the exploitation of this information. The communication (31) between the invention and the applications (314, 315, 316) and/or the objects (311, 312, 313, 317, 318, 319, 320) comprises wireless and encrypted communications.

In a preferred embodiment, the phase 1 phase of the data analyzer (71) comprises, but is not limited to, Logit models, Decision Trees, Neural Networks and other artificial intelligence classifiers to distinguish emotional states given the bio data.-marks received from the data store (62). The estimation of the emotion is sent to phase 2 of the data analyzer (72) for the analysis of future emotions.

In the stage of phase 2 of the data analyzer (72) it comprises, but is not limited to, artificial intelligence algorithms such as Hidden Markov Chains, Decision Rules, Bayesian Networks, Decision Trees and other artificial intelligence techniques to estimate the probability of future emotional state given the current emotional state as received from phase 1 of the data analyzer (71). The process of phase 2 of the data analyzer (72) also comprises of the logic necessary to communicate the current emotional state transmitted by the step of the phase 1 of the data analyzer (71) a stage comprising a phase 3 of the analyzer of data (73) where the data are adapted for the stage where the emotion state is modulated and predicted in the modulation and prediction block of the emotional state (74) which transmits the emotion to the module transmitter of the emotion (30) and at the same time send the data to the data store (62) for storage.

### Emotion Transmitter

This block includes the logic necessary to communicate the emotions that have been determined, as well as a prediction of the emotions of the users who are being monitored through the bio-transmitter (10) and whose current and future emotions have been established in the block (70) and can be transmitted to services, objects, software or other applications and/or services, based on World Wide Web or electronic interfaces (also known as WWW applications), existing or to be built that benefit from emotional information which is generated by means of this invention.

The emotion transmitter (30) comprises user interfaces (31) for portable electronic telecommunication devices that transmit a) the current emotional state of the user being monitored and; b) a prediction based on the current and historical emotional state for that user, which is obtained from the block (70). The emotional information resulting from the online processing system (20) is communicated to the emotion transmitter (30) by the information analysis module for estimating emotions and predictions (70) is carried out by means of wired and wireless communication protocols. This information allows WWW applications, services, objects or other applications and inventions to maintain the historical record, as well as the current emotions of the users and a prediction of the emotions of individual users in particular contexts or in various contexts in which it requires having a record of the emotions of individual users. In this way we have the recognition of emotional states and predictions of emotions of the monitored users.

In another preferred and particular embodiment, the Emotion Transmitter (30) comprises software and hardware components equipped with logical Internet location circuits through an IP (Internet Protocol) address integrated in objects or devices, including but not limited to video games, toys, automatic vending machines, household appliances, internet sales services, domotic products and/or services, as added functionality and through which current emotion is transmitted and prediction of future emotions (70) by what the object becomes aware of the emotional state of the user allowing the object or device to adapt or react according to these emotions (70).

FIG. 3 shows the block diagram of the system for monitoring, processing, analysis and storage of physiological signals for the recognition of emotions, as can be seen, at least one user of a plurality of users who has placed a plurality of sensors (01, 02, 03 and 04), but not limited to those represented here, placed in the places where the physiological signals will be monitored, which may be for example and not limited to monitoring an electro-dermal activity (EDA), heart rate (HR), blood pressure, temperature, neuronal activity and other physiological data, once captured said data, are sent to the user interface (11) of the mobile device, and by means of said interface is sent to the bio-transmitter block (10), and that as already mentioned above, through the Internet and wirelessly, this block communicates with the online processing system (20), which is in a service but not limited to this, for example, the online processing system (20) can also be in a computer system.

Once the processing of the emotion information of the at least one user in the system (20), which calculates the emotional variation, the result is sent to the mobile device, which can be the same mobile device or a different mobile device, that in the illustrated case, the data is sent to the same mobile device, the information is received by the module (30), which is the transmitter of emotions, in this way, once the information has been received, then the interface of user (31) is responsible for communicating the emotional states to other applications (314, 315, 316) or objects or devices (311, 312, 313, 317, 318, 319, 320) as described below in embodiments of the invention.

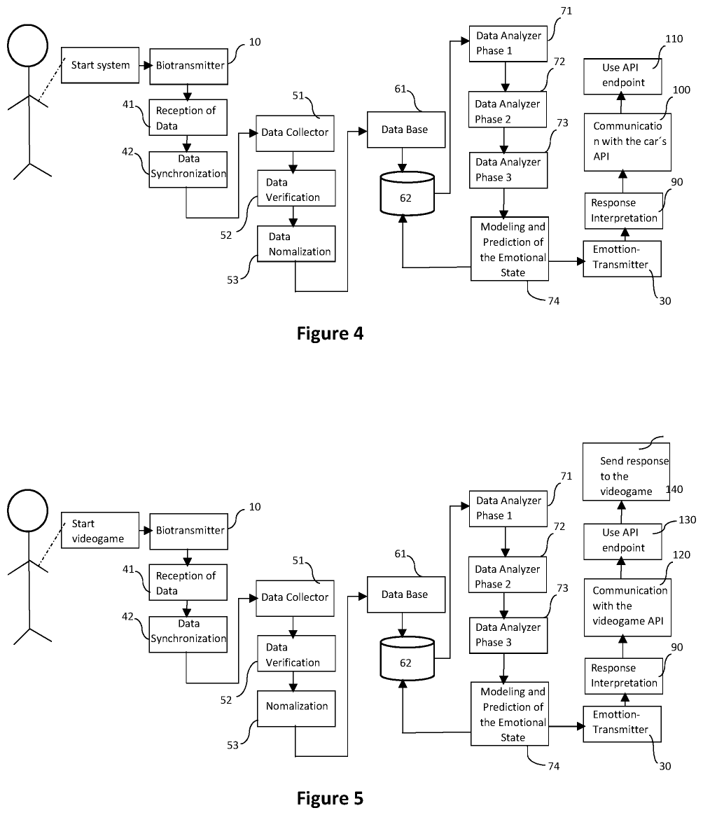

Taking into account FIG. 4, the modality shown is a preferred embodiment, so in this form of operation, when the bio-transmitter (10) takes the data of a user's physiological signals, wherein said bio-signals are originating in the sensors that can be electro-dermal activity (EDA) and heart rate (RC) and/or embedded in the steering wheel (01) of a vehicle (311), and where the user is a vehicle operator and then of having performed the processing of the data in the system (20), and having the emotional state of the operator which is sent to the emotion transmitter (30), then the mode of operation in this modality comprises at least but not limited to an electrical circuit with the logic necessary to be adapted to a vehicle, wherein the emotion transmitter (30) sends the current emotion of the driver of the vehicle, as well as a prediction of the driver's emotions, which is generated in the module (70).) of the system (20) and that is also received by the emotion transmitter (30) for its transmission to the vehicle, which is done through the user interface (31), in such a way that with the information that in this case receives the vehicle (311), where a response interpretation is made by means of the block (90), which is responsible for carrying out a new processing of the information in order to have a communication with the vehicle's API, this is done in the module (100), once the communication with the vehicle API is established, the use of the API EndPoint in the module (110) is carried out, in order to adapt the operating conditions and/or transmission of emergency messages based on the emotional state of the driver.

The vehicle driver may be operating a car for example, but it may also be a van, passenger bus, transport truck and other similar vehicles, when the emotion transmitter (30) receives the user's current emotional state, as well as the prediction of the emotional state of the user of the vehicle the transmitter of emotions (30) sends the information to the user interface (31) so that the vehicle (311) upon receiving said information and when processing it, alters the driving dynamics, communicates information to emergency systems or even disable the driving of the vehicle or adapt the speed of the vehicle to provide a safer driving experience for the driver of the vehicle (user) to which the vehicle is being monitored and adjusted based on his current and anticipated emotional state. it is generated in the module (70).

In this way, once the user interface communicates the emotional states of the operator of the vehicle, and when the automobile has processed said information, it can have an interpretation of the emotional variation in the user, then the dynamic alteration of driving consists, for example, if the driver of the vehicle encounters dangerous levels of fatigue or stress, alterations can be offered to the speed of the vehicle (311) to restrict it to safe speeds. Another alteration consists in warning using portable electronic telecommunications devices, to third parties that the driver is in a dangerous situation (312) due to negative emotional states (such as stress and/or extreme fatigue). Another alteration consists of giving spoken messages to the driver warning him of the dangers he encounters when driving in a state of stress and/or fatigue (313).

In FIG. 5 a preferred and additional mode of the system is shown, in this case the user interface (11) used to collect the bio signals and the user interface that communicates the emotional states (31) are carried out in a portable electronic telecommunication device. Through the user interface (11) physiological information is collected by means of the EDA and RC sensors embedded in a control of a video game, which in this case the sensor (01) is determined as the control of the video game, or by AN sensors (04) for a video game.

Once the readings of the bio signals are taken through the user interface (11), they are transmitted by means of the bio transmitter (10) to the online processing system (20), which calculates the emotional variation in the user and sends the result to the portable electronic telecommunications device or mobile device, this through the user interface that communicates the emotional states (31). In this way, once the application that communicates the emotional states (31) transmits to the video game the emotional variation in the user, the invention offers the possibility of continuing with the current development of the video game (314) or that the development is altered of the game depending on the emotional states, past, current and future (315, 316). Therefore, once the interpretation of the response of the module (90) has been performed, then the signal is sent to the module (120), in which a communication is established with the video game API, and subsequently in the module (130) the EndPoint of the video game API is used, already with the interpretation of the emotions that are read from the monitored user, with this information in the module (140) the appropriate response is sent to the video game.

An example of this modality would offer alterations considering non-player characters (NPC non-playing characters) that demonstrate emotions either equal or opposite to the user's emotions, automatic adjustments of the difficulty of the game or the inclusion of dramatic effects that they include elements of audio and video, characterizations, colors, musical tones, vibrations in the controller among others, designed in such a way that, technically, the invention has the capacity to modify the behavior of the video game, and as a result, the experience is improved emotional of the user.

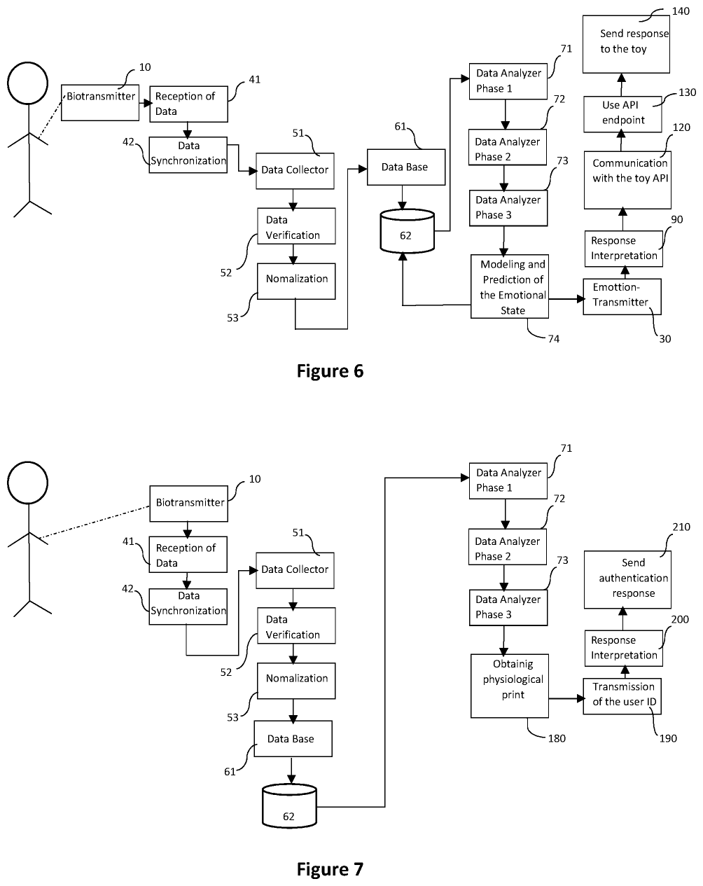

FIG. 6 shows one more mode of the system, which in this case is applied to toys, where the user interface (11) used to collect the bio signals of a monitored user and the user interface that communicates the emotional states (31) are carried out in a portable electronic telecommunications device or mobile device. Through the user interface (11) the physiological information is collected by means of the sensors (02, 03, 04), subsequently the user interface (11) transmits the bio signals by means of the bio transmitter (10) to the system online processing (20), which calculates the emotional variation in the user and transmits the result to the portable electronic telecommunications device through the user interface that communicates emotional states (31). In this way, once the user interface communicating emotional states (31) transmits to objects or devices comprising animals and/or stuffed animals, they can automatically pronounce emotional words (318), move their hands emotionally (319) , change color (320), or other tactile movements or emotional effects that the toy could have depending on the emotional state of the user.

As seen from FIG. 6, once the calculation of the emotion of the monitored user has been made, the signal that is sent by the emotion transmitter (30) and received by the module that performs the interpretation of the response (90)), a communication is carried out with the API of the toy in the module (150), that once the communication is established and according to the interpretation of the emotion, then in the module (160) the EndPoint of the API of the toy, so that in this way the toy by means of the module (170) can perform the behavior of the toy as already described above.

In FIG. 7, a preferred additional embodiment of the invention is shown, the user interface (11) for collecting the bio-signals of the monitored user and the user interface that communicates the emotional states (31) are carried out in an electronic device. portable electronic telecommunications or mobile device. Through the user interface (11) the physiological information is collected by means of at least the sensors (02, 03). The user interface (11) transmits the bio signals by means of the bio-transmitter (10) to the online processing system (20), which calculates the emotional variation in the monitored user and transmits them to the portable electronic telecommunications device. through the emotion transmitter (30) and through the user interface (31) the emotional states of the monitored user are communicated.

In this modality, once the user interface (31) is ready to communicate the emotional states of the user, it communicates them to automatic teller machines (ATM) or cash machines (317), a way to maintain security, the invention contains incorporated logical circuits so that the user provides a physiological fingerprint, which is generated by means of the physiological fingerprint obtaining module (180) contained in this modality in the block (70), this physiological fingerprint forms a third piece of information, since that together with your bank card (as an element that the user provides) and your PIN number (as an element that the user knows) are combined to obtain the identity of the user (ID), in this way the user's ID transmitter module (190)) transmits the identity of the user so that the device (317) can identify the user, so that the response interpretation module (200) is responsible for performing the interpretation of the information received, and thus, in the authentication response sending module (210) the identity of the user can be sent to the device (317) and thus, allow the user to operate the device (317),

Under this perspective, the ATM (317) asks the user to show a unique, relevant and true emotional state for that user and which can only be provided to the cashier through the application that communicates emotional states. This security measure can be extended to any object, device or device that requires user authentication, including vehicles, which could turn on the engine only if the user has the proper physiological fingerprint.

Other objects or devices that could alter their behavior using the emotional state, include but are not limited to appliances, computers, home automation devices and any other object or device capable of accessing the Internet from where you can receive emotional information from the user interface (31) that communicates the past, present and future emotional states of the user, interacting with the object in question and in which the behavior of this object can be altered using the emotional information and adapting to the desired behavior and specified by the user.

In additional mode, the user interface (11) collects physiological information by means of EDA and RC sensors and transmits the bio-signals to the online processing system (20), which calculates the emotional variation in the user and the result is sent to the portable or mobile electronic telecommunications device through the emotion transmitter module (30) and through the user interface (31) the emotional states are communicated to the connected devices, which could be, for example, a platform in a work center, in such a way that the invention offers the possibility of continuing with the current activity of the user without altering their work, in such a way that in this modality the personnel manager is offered the emotional information of the users without affecting their anonymity, in order to demonstrate the emotions at a global level of the company where the invention is used.

In a further modality, the outputs (past, present and future emotions) of the online processing system (20) comprise past, present and future emotional states required by the emotion consultation system that are stored in an information storage medium, which is in a data warehouse (60). This communication required by the user through standard WWW interfaces is supported by the Internet and allows the user to track their emotions in real time at any time and in association with any activity.

In this modality, the emotional consultation system takes the form of an online service, in which the user can track their emotional states in specific activities such as driving a vehicle, playing a video game and other specific activities that use this invention. Additionally, the emotion consultation system can also take the form of an application for a portable electronic telecommunications device where the user can also track their emotional states, in this way, the applications, objects, devices or services, as well as the user can access the emotional states generated by the users at all times. The emotion consultation system may also comprise tools that allow the user to create graphs of their emotional changes during a particular period of time or during interaction with a particular object, device or service, or any other electronic device or web-based application. , which includes, but is not limited to social networks, health, work performance, military, or any application where emotional information can be exploited.

In alternate modes, the bio-transmitter (10) can also take the form of an integrated chip which can be installed on personal items such as electronic watches, lenses, contact lenses, rings, or any other personal object that the user uses. Carry it on a regular basis.

The emotion transmitter (30) can also take the form of an integrated chip which can be installed on any object, toy, computers, vehicles, appliances, home automation devices, furniture, clothing, or any other object or device with an internet connection. that can exploit the emotions of users both past, present and future.

In an additional modality, the system collects information on Neural Activity (NA) read through a neuronal activity reading device (04) of a monitored user, Electro Dermal Activity (EDA). is read using an electrodermal activity reading device (02) and the Heart Rate (RC) read by a Heart Rate reader device (03), are collected by the user interface (31) of a portable electronic telecommunications device. The bio-transmitter (10) is designed with the necessary logic to encrypt the signal and sends it through the internet to the online processing system (20) located in a centralized server and permanently connected to the internet. The receiver (41) is designed to receive encrypted information, it also contains the logic necessary to decrypt the information sent by the bio-transmitter (10) and to separate the information into components. In the best mode of operation, the data includes EDA, RC and NA.

Since the typical reading frequencies for EDA, RC and NA are different, these signals must be synchronized so that they are expressed in the same timeline, therefore, the receiver (41) sends the separated information in these two components to the data synchronization module (42), in which the data synchronization step is performed, for this, the module (42) has the necessary logic for, for example, averaging 8 EDA readings (assuming a frequency of 8 data per second) in only one reading per second. The purpose of doing this is to allow the system to standardize the bio-signals since they may have different frequencies.

When the bio-signals are processed in the module (42) and are normalized, then the signals of EDA, RC and NA already in the same timeline (one data per second, for example) are sent to the collector module of data (51) which in turn puts the information in an appropriate data structure. For example, EDA may consist of information expressed in non-integer format while NA may consist of information expressed in integer format. The stage carried out in the data collector module (51) comprises algorithms that identify the bio-signals and arrange them in a data structure corresponding to the type of data that is entered. Subsequently, the information already located on the same timeline and in the correct format is sent to the stage of the data verification module (52), together with the unique user identifier (UUI), which verifies that the information received from the data collector (51) does not include lost or erroneous information. For example, a reading of the letter “P” when a whole number is expected would mark this reading as erroneous. A correct reading, for example “1” when a whole number is expected, would mark this reading as appropriate. Correct and erroneous readings are passed along with their corresponding UUI to the normalization module (53).

The normalization module (53) of the bio-signals comprises the realization of information transformations, the completion of erroneous information and other information normalization techniques to present the information in the correct format. For example, given that the frequency of EDA information is 8 data per second, if an erroneous mark is read for this record it may be the cause of information loss, for example, only 4 readings instead of 8. This loss of information may be due to the loss of communication by the bio-transmitter (10), the stage performed in the normalization module (53) of the bio-signals comprises algorithms that use parameters to estimate the loss of information. If there is a visible correction, at this stage the error is corrected and the information is normalized by averaging 4 pieces of information instead of 8, at the end of the process, only the correct information is sent to the information storage stage in the warehouse. data (60).

The information storage system (61) handles the information that comes from the standardization module (53) of bio-signals and organizes the information in a database structure. For example, an index for the record is provided which contains information EDA, RC, NA and the unique user identifier (UUI), also adding the arrival time to the storage system (61) as well as the context in which information is gathered for later storage in the data warehouse (62). In this way, a record for the database includes the relevant information that is being read (EDA, RC, NA and UUI in this example) as well as the time of arrival and the context in which the information is being generated, by example, while driving a vehicle.

The data warehouse (62) comprises a commercial database administrator system based on SQL protocols. By using this commercial application, it becomes routine to store information from the information storage system (61) and subsequently, to administer the query of data made by Phase 2 of the information analyzer (72) and the emotional query system. In the best way of operation, the information can be stored for one or several users at the same time. Once the record contains EDA, RC, NA and the UUI is stored in the appropriate place for a user, the data store (62) passes the record containing the following fields: time, NA, EDA and the UUI to the Phase 1 of the data analyzer module (71).

Phase 1 of the data analyzer (71) uses Artificial Intelligence that includes, but is not limited to the use of Probit models, logistic models, Decision Trees, Neural Networks and other classification algorithms to determine the emotional state by which the user is happening in the registration time and context (for example, the driving of a vehicle or the interaction with a video game, toy or other object, product or service) also specified in the record. As a result, Phase 1 of the data analyzer (71) comprises an emotional state based on the bio-signals, the time and the specific context in the record that is sent by the data warehouse (62). This emotional state together with the registration thereof, is sent to Phase 2 of the data analyzer (72).

Phase 2 of the data analyzer (72) when receiving the data performs the second stage of the process to analyze the information for which it uses Artificial Intelligence algorithms that include, but are not limited to the use of Markov Hidden Chains, Bayesian Networks, Decision Trees and other Artificial Intelligence techniques to estimate a future emotional state. Phase 2 of the data analyzer (72) consults the data store about past emotional records for this user. These emotions are used by Artificial Intelligence algorithms to estimate the most likely future emotion. Then this emotion is passed on to the emotion transmitter (30) along with the current emotion, and the database administrator is instructed to store the prediction the correct record.

Once the emotion transmitter (30) has the information, it uses logic circuits and applications for a portable electronic telecommunication device in which a user interface (31) will indicate the user, object, device, product or service that requires it, the most likely emotional state given the current circumstances and past emotional experiences for the same user. Also, in the best mode of operation and in a preferred embodiment, the user interface (31) of the portable electronic telecommunication device communicates the current and future emotional state to other inventions and applications in relation to the context for example during the driving of a vehicle or the use of a video game. In this way you have the adaptability of the system in a vehicle, for example, but not limiting to it, as already mentioned.

Considering the above description, in an additional modality, there is a computer readable medium, in which the information containing the steps of a method to monitor, analyze and store physiological signals for the recognition of emotions, the steps is stored. of the method perform the steps widely described in the present technical specification.

What has been described and illustrated in this document are preferred embodiments of the invention along with some of its variants. The terms, descriptions and figures used in this document are set forth as illustration only and do not necessarily limit the preferred embodiment to them. Those skilled in the art in connection with this invention will recognize that many variations are possible within the scope of this invention, in which all terms are referred to in their broadest and most reasonable sense unless otherwise indicated. All titles used in the description are for convenience only and have no legal or limiting effect.

1. A system for the monitoring, processing, analysis and storage of physiological signals for the recognition of emotions, the system comprising:
at least one sensor, of a plurality of sensors, which monitor at least one user of a plurality of users, physiological signals (bio-signals) to determine the emotional state of said user;
a first user interface, within a mobile device, which is responsible for receiving the bio-signals taken by said users from the at least one sensor of the plurality of sensors;
a bio transmitter that receives the bio signals of the first user interface with the information of the bio signals of the at least one user of the plurality of users, the bio transmitter performs a processing of the bio-signals to subsequently transmit said bio signals;
an online processing system that is located on a server and that receives the bio signals from the bio transmitter, the online processing system is responsible for carrying out the analysis of the information contained in the bio signals and in based on said information, the emotional state of the at least one user of the plurality of users who are monitored in real time is determined, so that based on the result obtained from said processing the condition of said at least one user is determined;
a transmitter of emotions, which is responsible for receiving the result obtained from the online processing system, by means of it to inform the emotional state of said user;
a second user interface, which is located inside a mobile device, and which receives the information of the emotional state of the at least one user, by means of the transmitter of emotions, so that through this user interface said emotional state is communicated in real time to at least one device, an object, an application or another system, in order to perform the corresponding action in said device, object, application or system depending on the emotional state of said user, allowing the device, object or user receiving the emotion becomes aware of the emotional state of the user, allowing the object, device or user to react according to these emotions.

2. The system for the monitoring, processing, analysis and storage of physiological signals for the recognition of emotions as in claim 1, wherein the plurality of sensors monitor physiological signals such as neuronal activity, electro-dermal activity, heart rate, blood pressure, temperature.

3. The system for monitoring, processing, analysis and storage of physiological signals for the recognition of emotions such as that of claim 1, wherein the bio-transmitter communicates with the online processing system wirelessly via internet.

4. The system for monitoring, processing, analysis and storage of physiological signals for the recognition of emotions such as that of claim 1, wherein the bio-transmitter communicates with the online processing system in a wired manner by means of Internet.

5. The system for monitoring, processing, analysis and storage of physiological signals for the recognition of emotions as in claim 1, wherein the bio-transmitter performs an encryption of the bio signals before transmitting said information.

6. The system for monitoring, processing, analysis and storage of physiological signals for recognition of emotions as in claim 1, wherein the bio-transmitter compresses the data before being transmitted to the online processing system.

7. The system for monitoring, processing, analysis and storage of physiological signals for the recognition of emotions such as that of claim 1, wherein communication between the bio-transmitter and the bio-sensors is carried out by means of the first interface of user and link is wireless, being able to increase or reduce the number of bio-signals used to increase or decrease the number of sensors.

8. The system for monitoring, processing, analysis and storage of physiological signals for the recognition of emotions such as that of claim 1, wherein the mobile device can be, for example, an intelligent cellular telephone.

9. The system for the monitoring, processing, analysis and storage of physiological signals for the recognition of emotions as in claim 1, wherein the online processing system has a bio-signal receiver that is responsible for receiving the signals which arc sent by the signal bio transmitter, so that once received, they are sent to a signal cleaner and producer of information, which is responsible for collecting the data, to perform a verification of the data and normalize the signals, to send the data to a data storage, and thus keep the information available, then the information is sent to an information analysis module, where the emotional state of the user is determined for a particular situation or circumstance and produces a prediction for the user or particular circumstance.

10. The system for monitoring, processing, analysis and storage of physiological signals for the recognition of emotions as in claim 1, wherein the bio-signal receiver has a signal receiving module which is responsible for receiving the bio - signals from the bio-transmitter, and once the bio-signal is received, it is classified according to its origin, such as heart rate, neuronal activity, blood pressure, and other physiological signals, to be sent later to a data synchronization module, where the data of the bio-signals are synchronized in a homogeneous time scale, reflecting a unique and constant change over time for each user.

11. The system for the monitoring, processing, analysis and storage of physiological signals for the recognition of emotions as in claim 1, wherein the bio signal cleaner has a bio signal collector that receives the signals from the synchronization module of data to organize again the bio signals but now in the same timeline, a module of verification of bio signals that is in charge of verifying the bio signals that it receives from the collector module of bio signals where it is verified that the received information is significant and enough to be normalized, a standardization module, where the bio signals of the verification module are received to organize the data in information packets where the bio signals that are being processed are associated to a Unique User Identifier (UUI).

12. The system for the monitoring, processing, analysis and storage of physiological signals for the recognition of emotions such as that of claim 1, wherein the data storage receives the standardized bio signals and their UUI identifiers, which allows to classify and organize the information of the processed data and thus store them in the system database.

13. The system for the monitoring, processing, analysis and storage of physiological signals for the recognition of emotions as in claim 1, wherein the information analysis module associates the data of the bio signals as they were received from the data storage , and the UUI to estimate the current emotional state and to produce a prognosis based on said emotional state, also considering past emotions, which are taken from the storage of data, and which are associated with a particular user.

14. The system for the monitoring, processing, analysis and storage of physiological signals for the recognition of emotions such as that of claim 1, wherein the transmitter of emotions, which receives the emotional state in real time, as well as a prediction of emotions future, which is inside a mobile device that can be for example a smartphone.

15. The system for monitoring, processing, analysis and storage of physiological signals for the recognition of emotions such as that of claim 1, wherein the device or object can be a car, cargo truck or any other vehicle that is driven, the driver being the user that is being monitored and to which the current emotional state of the driver can be determined as well as an emotional prediction of the driver, in such a way that the second user interface sends the data to the vehicle, to know if the driver is find tired, stressed or in any condition that puts the operation of the vehicle at risk, so in this case, the vehicle is aware that the operator is not in driving condition, so that the vehicle adapts the driving conditions and/or make the transmission of means emergency services that are based on the emotional state of the driver, as well as send messages to the driver to communicate the risk in which he is driving in that state.

16. The system for the monitoring, processing, analysis and storage of physiological signals for the recognition of emotions such as that of claim 1, wherein the device or object can be a video game, where the sensors can be embedded in the control of the video game, in such a way that the video game responds to the emotions in which the user finds himself.

17. The system for the monitoring, processing, analysis and storage of physiological signals for the recognition of emotions such as that of claim 1, wherein the device or object can be a toy that can respond according to the emotional state of the user, where such response may be, for example, moving hands, pronouncing words, changing colors, or other tactile movements or emotional effects that the toy might have depending on the emotional state of the user.

18. The system for the monitoring, processing, analysis and storage of physiological signals for the recognition of emotions such as that of claim 1, wherein the device or object can be an ATM (ATM) or cash machines, wherein the ATM can request the user to show a unique, relevant and true emotional state for that user and which can only be provided to the cashier through the application that communicates emotional states.

19. The system for the monitoring, processing, analysis and storage of physiological signals for the recognition of emotions such as that of claim 1, wherein the device or object that can alter its behavior can be a household appliance, computers, home automation devices, and any other object or device that can access the Internet.

20-35. (canceled)
